(19) United States
(12) Patent Application Publication
(10) Pub. No.: US 2025/0161026 A1
(43) Pub. Date: May 22, 2025

(54) Lens with Extended Depth of Focus by Inducing an Excess of Longitudinal Chromatic Aberration

(71) Applicants: Gerd Auffarth, Schriesheim (DE); Grzegorz Labuz, Dossenheim (DE)

(72) Inventors: Gerd Auffarth, Schriesheim (DE); Grzegorz Labuz, Dossenheim (DE)

(73) Assignee: Universität Heidelberg, Heidelberg (DE)

(21) Appl. No.: 19/036,179

(22) Filed: Jan. 24, 2025

Related U.S. Application Data
(63) Continuation of application No. PCT/EP2023/070866, filed on Jul. 27, 2023.

(30) Foreign Application Priority Data
Jul. 27, 2022 (EP) 22187235.1

(51) Int. Cl. A61F 2/16

(52) U.S. Cl. CPC A61F 2/1659; A61F 2/164; A61F 2/1656

(57) ABSTRACT

Inter-alia, an implantable lens is disclosed comprising a refractive element causing chromatic aberration and an element inducing an increase of the chromatic aberration. It is further disclosed a method for producing an implantable lens.

### CROSS-REFERENCE TO RELATED PATENT APPLICATIONS

This patent application is a continuation of International Application No. PCT/EP2023/070866, filed on Jul. 27, 2023, which claims the benefit of priority to European Patent Application No. 22187235.1, filed Jul. 27, 2022, the entire teachings and disclosures of both applications are incorporated herein by reference thereto.

### FIELD OF THE INVENTION

The following disclosure relates to the field of implantable lenses, e.g. intraocular lenses (IOLs) or phakic lenses, in particular to implantable lenses that induce an excess of longitudinal chromatic aberration (LCA) to extend the depth of focus (DoF) of an eye after e.g. crystalline lens removal.

### BACKGROUND OF THE INVENTION

The treatment of eye disorders may involve the removal of the natural lens, e.g., in cataract surgery. In these cases lost optical power is usually replaced with an intraocular lens that may be implanted at the position of the former natural lens. Other cases of treatment of eye disorders may involve leaving the natural lens intact but e.g., correcting high refractive errors by using a phakic lens. Conventionally, a monofocal intraocular lens may be utilized for eye disorder treatment that converges the light approximately into one focus, which determines the point at which a patient can see clearly. However, objects located either in front or behind the focal plane are blurred, and thus, cannot be recognized by a patient. Alternatively, multifocal lenses, e.g., bifocal or trifocal lenses, are used, such as described e.g. in US 2014/0168602 A1, that extend the depth of focus by introducing more than one focal plane. These e.g., split incoming light into primary and secondary foci, resulting in decreased visual quality due to the energy distribution. Also, lenses having standard zonal designs may exhibit a dependency on the individual patient's pupil. Usually, either monofocal or multifocal lenses are configured to provide reduced chromatic aberration as chromatic aberration is generally believed to be an effect detrimental to the imaging quality of a lens.

Patent application US 2019/0110889 A1 considers a combined refractive and diffractive structure to create a hyperchromatic lens, wherein the refractive structure has a positive power.

European Patent 83305354.9 discloses an ophthalmic lens having a refractive and a diffractive power, wherein the diffractive power is provided by a transmission/surface relief hologram.

US 2010/0312337 A1 and US 2014/0168602 A1 propose ophthalmic lenses that apply a diffractive profile to reduce LCA.

### SUMMARY OF THE INVENTION

It is therefore an object of the invention to propose an implantable lens that overcomes the drawbacks of the known lenses and in particular to propose an implantable, e.g., an intraocular lens, providing an extended depth of focus that addresses the needs of pseudophakic patient, e.g., after cataract surgery, in particular with respect to enhancing a patient's range of vision with minimal side effects.

According to a first example aspect, this object is solved by an implantable lens comprising a refractive element causing chromatic aberration and an element inducing an increase of the chromatic aberration, wherein the element inducing the increase of the chromatic aberration comprises a diffractive structure, wherein the diffractive structure is a negative diffractive structure and wherein the diffractive structure is a Kinoform lens structure. So, the diffractive structure may be a negative or diverging Kinoform lens structure, as such it may exhibit or yield a negative focal length and may diverge incident light rays. Hence, it may increase LCA as a Kinoform structure acting as a negative lens. A negative or diverging Kinoform lens structure may reduce the base power of the refractive component, in particular between −3 and −8 D, e.g. −3.75 D or −7.25 D.

As an example, an implantable lens is disclosed comprising a refractive element causing chromatic aberration and an element inducing an increase of the chromatic aberration, wherein the element inducing the increase of the chromatic aberration comprises a diffractive structure to diverge the incident rays having a negative focal length, wherein the diffractive structure is a Kinoform lens structure. As a further example, an implantable lens is disclosed comprising a refractive element causing chromatic aberration and a diffractive element inducing an increase in chromatic aberration (of the refractive element), wherein the diffractive element inducing the increase of the chromatic aberration comprises a negative diffractive structure and wherein the diffractive structure is a Kinoform lens structure.

An implantable lens may be, e.g. an ophthalmic lens, intraocular lens (IOLs), pseudophakic lens or a supplementary lens. An implantable lens may be understood as an intraocular lens (IOL) for (e.g. capsular-bag) implantation, or as a supplementary sulcus-fixated lens or a phakic lens. An implantable lens and/or a refractive element may have a size (e.g. a diameter) of 5.5 to 7 mm (e.g. 6 mm).

A phakic lens may be a phakic intraocular lens which may be understood to be an intraocular lens that is implanted surgically into the eye to correct myopia (i.e. near-sightedness). A phakic lens may be utilized in case the natural lens is left intact in an eye.

An intraocular lens may be understood to be a lens implanted in the eye e.g. as part of a treatment for cataracts or myopia. Such an IOL may e.g. be implanted during cataract surgery, after an eye's natural lens having e.g. a cataract has been removed. A pseudophakic IOL aim to mimic the same light-focusing function as the natural crystalline lens.

A phakic intraocular lens which may be placed in the anterior chamber to function with the existing natural lens and may e.g. be used in refractive surgery to change the eye's optical power e.g. in case of a treatment for myopia. A supplementary lens may be understood to be a lens that is implanted in an eye, e.g. in the sulcus, along with another lens, e.g. a standard in-the-bag lens. Also, a supplementary lens may have different geometry than an IOL. For instance, a refractive element of an IOL may be biconvex, i.e. the posterior and anterior surface are both convex. Whereas, a refractive element of a supplementary lens may have one surface convex and the other concave, e.g. the refractive element may have a converging meniscus or a negative or positive meniscus.

An implantable lens or IOL according to the first aspect may comprise or consist of a small (e.g. plastic) lens with (e.g. plastic) side struts, e.g. haptics, to hold the lens in place in a capsular bag inside an eye. Haptics may have a rounded or spiral form that may allow for a precise centration and fixation within an eye. An IOL may e.g. be placed in an anterior chamber or posterior chamber or within a capsular bag e.g. during cataract surgery. For instance, during surgery, a cataractous lens may be removed from its capsular bag and replaced by an implantable lens according to the first example aspect. The implantable lens according to the first example aspect may comprise or consist of (or made of) a foldable material (i.e. a material that can be folded), which may enable insertion into an incision of up to 4 mm diameter and may minimize any damage which may be caused by the surgery and postoperative scarring. For instance, the implantable lens according to the first example aspect may comprise or consist of (or made of) a plastic such as polymethyl methacrylate (PMMA), hydrophobic acrylic (e.g., phenylethyl methacrylate (PEMA) and phenylethyl acrylate (PEA)); hydrophilic acrylic (e.g., poly hydroxyethylmethacrylate (pHEMA)); and/or silicone (e.g., poly dimethylsiloxane (PDMS)), which may prevent postoperative complications associated with scarring.

The sulcus may be the ciliary sulcus which may be understood to be a small space between the posterior surface of the iris base and the anterior surface of the ciliary body. The sulcus may be utilized to fixate an implantable lens according to the first example aspect, which may provide a good long-term stability and safety.

A capsular bag may be understood as a sack-like structure remaining within the eye following extracapsular cataract extraction or phacoemulsification. An implanted (intraocular) lens may be placed within this structure to recreate the (original) phakic state.

A refractive element causing chromatic aberration may be an optical element that causes refraction and exhibits (longitudinal) chromatic aberration (LCA). Chromatic aberration may be caused by dispersion within the refractive element, e.g. the refractive index of the elements of a lens may vary with the wavelength of incoming light. Since the focal length of a lens may depend on the refractive index, a variation in refractive index may affect focusing. A refractive element causing chromatic aberration may be a lens, e.g. a biconcave lens. It may be a monofocal or multifocal implantable lens, e.g., an extended-depth-of-focus, bifocal or trifocal lens. A refractive element may have a base (i.e. intrinsic) LCA of up to (about) 1 D or may have a base LCA of less than 0.25 D. LCA may be determined as the difference between a refractive element's, e.g. a lens', nominal powers measured at the extreme ends of a light spectrum (e.g., 450 nm and 650 nm), e.g.

$$L C A = P ( {λ}_{2} ) - P ( {λ}_{1} )$$

wherein λ₁ and λ₂ may be different light wavelength (e.g., 450 nm and 650 nm), for a diffractive element, the LCA may depend on a design wavelength λ₀, e.g. 550 nm, and P₀ the (refractive) power of the diffractive element at wavelength λ₀. LCA may e.g. be measured by measuring P(λ₁), P(λ₂), or estimated based on the wavelength. The power in diopters (D) may be the inverse of the refractive elements, e.g. the lens', focal length measured in a specific medium (e.g. medium with n=1.336 such as an aqueous humour, e.g. when measured in vivo, in particular in situ e.g. in the eye, or a balanced salt solution, e.g. when measured in vitro, in particular in immersion) with a given refractive index. (Longitudinal) chromatic aberration may depend on the Abbe number of the used material(s). The Abbe number or V-number of a (transparent) material, is an approximate measure of the material's dispersion (i.e. the change of refractive index versus the wavelength of incoming light), high values of V may indicate low dispersion. An (IOL) refractive element may have an Abbe number of 30 to 60, in particular 40 to 50, e.g. 42 or 46, e.g. it may be made from a material with an Abbe number of 30 to 60, in particular 40 to 50, e.g. 36, 42, 46, or 55. A refractive element may have base (refractive) power or nominal power range of 0D to 40 D, in particular 10 D to 30 D, e.g. 20 D to 30 D. A refractive element causing chromatic aberration may have a central thickness of up to 2 mm, e.g. 1 mm, wherein a central thickness may be the thickness of the refractive element along its optical axis, e.g. the thickness of the refractive element along the axis about which a refractive element may be rotational symmetric, e.g. in case of a refractive element being a lens. A refractive element may have a refractive index of more than 1, preferably at least 1.01 or at least 1.3 and/or at most 3, more preferably at least 1.4 and/or at most 2, in particular (about) 1.5, e.g. 1.49 or 1.52; e.g. the refractive element may be made from a material having a refractive index of more than 1, preferably at least 1.01 and/or at most 3, more preferably at least 1.3 and/or at most 2, in particular (about) 1.5, e.g. 1.49 or 1.52. A refractive element may have a refractive index higher than the refractive index of the eye's median, e.g. higher than 1.336. Stated refractive indices are with respect to a light wavelength of 589 nm. A refractive element causing chromatic aberration may be made from an optical material (e.g. a biomaterial), e.g. an amorphous or single crystalline material, such as PMMA, silicone, acrylate. For instance, a refractive element may comprise or be made from e.g. polymethyl methacrylate (PMMA), hydrophobic acrylic (e.g., phenylethyl methacrylate (PEMA) and phenylethyl acrylate (PEA)); hydrophilic acrylic (e.g., poly hydroxyethylmethacrylate (pHEMA)); and/or silicone (e.g., poly dimethylsiloxane (PDMS)). The refractive element may exhibit a convex surface (structure) it may e.g. be biconvex, e.g. a biconvex lens. In particular the refractive element may cause a chromatic aberration with a distance between the foci for red and blue light of 1 to 2 mm, as measured in an immersion (as e.g. described above), and/or 0.4 to 0.6 mm, as measured in situ or in the eye.

An element inducing an increase (or a surplus, an excess, or an excessive amount) of the chromatic aberration may be an element that increases the chromatic aberration with respect to the (intrinsic) chromatic aberration of e.g. a refractive element, e.g. the chromatic aberration exhibited by the refractive element. Chromatic aberration may be longitudinal chromatic aberration (LCA). For instance, an element inducing an increase of the chromatic aberration may be an element increasing the longitudinal chromatic aberration. The (longitudinal) chromatic aberration may be increased by e.g. 1 D, 2 D, 3 D, 3.5 D or more to about 4 D, in particular for a light wavelength of 450-650 nm. For instance, LCA may be dependent on materials and (optical) powers of the implantable lens and/or refractive element and/or element inducing an increase of the chromatic aberration. An increase of LCA may be an increase by a certain value (e.g. from a base power of 0.25 to 3 or 4D). For instance, an element inducing an increase of the chromatic aberration may increase the distance between foci for different light wavelength, e.g. by stretching the foci for different light wavelength further apart, it may e.g. increase the distance between a focus (e.g. a focal plane) for red light (e.g. a wavelength of 650 nm) and for blue light (e.g. a wavelength of 450 nm) e.g. dependent on the lens power of the refractive element and its Abbe number. For instance, the distance between a focus for red light and for blue light may be increased, under standard room conditions (e.g. a temperature of 20° C. (293.15 K), an absolute pressure of 1 atm (101.325 kPa), dry air atmosphere), to at least 5 or 6 mm or at least 9 mm, it may be increased e.g. to 6 to 12 mm or 13 mm, e.g. about 6.8 or 11.4 mm. Preferably, the distance between a focus for red light (e.g. 656 nm) and for blue light (e.g. 486 nm) may be between 5 mm and 10 mm as measured in immersion, wherein the immersion has a 1.333 refractive index (i.e. of the surrounding medium of the lens during measurement) and/or the distance between a focus for red light (e.g. 656 nm) and for blue light (e.g. 486 nm) may be between 0.8 mm and 1.3 mm as measured in situ (e.g. in the eye). So, the chromatic focus shift between 656 nm and 486 nm may be 5 to 10 mm in immersion and 0.8 to 1.3 mm in situ. For comparison, the chromatic shift between 656 nm and 486 nm of the refractive element may be about 1 to 2 mm (in immersion) and/or about 0.5 mm in situ. For instance the refractive element may cause a chromatic aberration with a distance between the foci for red and blue light of 1 to 2 mm, as measured in the immersion, and/or 0.4 to 0.6 mm, as measured in situ. An element inducing an increase of the chromatic aberration may comprise or consist of (e.g. be made of) any suitable material (for instance a biomaterial), e.g. any of the materials described in the context of a refractive element causing chromatic aberration, e.g. the same material(s) or biomaterial(s) as a refractive element causing chromatic aberration. For instance, the refractive element causing chromatic aberration and the element inducing an increase of the chromatic aberration may comprise or consist of (e.g. be made of) the same material or a different material. An element inducing an increase of the chromatic aberration may be attached to, placed on or formed onto a refractive element's posterior or anterior surface. An element inducing an increase of the chromatic aberration may have a back surface being congruent with a refractive element's posterior or anterior surface or it may be smaller, e.g. limited to the central-lens area, e.g. 3 mm to 4 mm in diameter. The latter may further allow improved mesopic and scotopic vision.

Contrary to the standard approach to minimize chromatic aberration it has surprisingly been found that increasing the chromatic aberration using an element inducing an increase of the chromatic aberration may be beneficial e.g. in extending the depth of focus with respect to a monofocal lens e.g. for treatment of pseudophakic patients or for treatment of presbyopia without removal of the natural lens. For instance, an increase of the chromatic aberration may provide an elongated focus shift, which may lead into extending patients' visual range in polychromatic light. Thus, an implantable lens according to the first example aspect may elongate the focus by incorporating various light wavelengths from the visible range to enable better vision at different distances in patients after cataract surgery.

An implantable lens according to the first example aspect may further be fully pupil independent, which is not feasible to achieve in e.g. refractive enhanced monofocal lenses, which may use the central portion of the lens to induce defocus. An implantable lens according to the first example aspect may further be more tolerable to postoperative refractive surprise due to the stretch of the polychromatic focus. An implantable lens according to the first example aspect may, hence, enhance a patient's range of vision while minimizing side effects associated with a standard approach of light split. Also, an implantable lens according to the first example aspect may be more cost-effective, e.g. more cost-effectively produced than e.g. a multifocal lens, for instance because more cost-effective materials e.g. with lower Abbe numbers, can be used.

According to a first example embodiment of the implantable lens according to the first example aspect the increase of the chromatic aberration is an increase by a factor of at least two, preferably at least three, in particular a factor between three and seven (e.g. in situ or in immersion). It has been found that a higher increase of chromatic aberration may be preferable to enhance the achieved effects. However, an increase over a factor of 7 may not be feasible with cost-effective materials. The factor may be a factor compared to the chromatic aberration caused by the refractive element, e.g. caused only be the dispersion of the refractive element, e.g. a refractive lens base, which may be measured in immersion.

According to a second example embodiment of the implantable lens according to the first example aspect the refractive element has a posterior and anterior surface, the posterior and/or the anterior surface having an aspheric or a spherical shape. An aspheric or a spherical shape may enhance the optical properties of the refractive element and may allow for precise adaptation of the refraction of the refractive element and may further enhance a patient's range of vision while minimizing side effects. A spherical shape (e.g. surface profiles of a refractive element, e.g. a lens, are portions of a sphere) or a cylindrical shape (e.g. surface profiles of a refractive element, e.g. a lens, are portions of a cylinder) may be easier and more cost-effective in production. An aspheric shape (i.e. not spherical shape, e.g. lens whose surface profiles are not portions of a sphere or cylinder) may avoid, reduce, or eliminate spherical aberration and hence, further enhance a patient's range of vision while minimizing side effects.

An element inducing an increase of the chromatic aberration may have an Abbe number of 30 to 60, in particular 40 to 50, e.g. 42 or 46, e.g. it may be made from a material with an Abbe number of 30 to 60, in particular 40 to 50, e.g. 36, 42, 46, or 55.

The posterior and anterior surface may be defined by the direction of incoming light traveling from the outside into an eye, when the lens is implanted such that the posterior surface is the surface first passed by the incoming light, i.e. the surface facing the outside or the cornea, when the lens is implanted, whereas the anterior surface faces the inside of the eye, e.g. the vitreoretinal chamber or retina, when the lens is implanted.

According to a third example embodiment of the implantable lens according to the first example aspect the element inducing the increase of the chromatic aberration comprises a diffractive structure, in particular the diffractive structure is a Fresnel structure, in particular a Kinoform lens structure.

A diffractive structure may be an optical structure, e.g. specifically designed for, inducing diffraction of incoming light. A diffractive structure may utilize the first and the second diffractive orders, and/or (e.g. partly) the 3rd diffractive order. A first and/or second diffractive order may be positive or negative, i.e. have a positive or negative sign, respectively. A preferable example of a diffractive structure may be a Fresnel structure or more preferably a Kinoform lens structure, which may allow for more cost-effective manufacturing and enhanced adaptability to patient's needs. It may allow further enhancement of the increase in chromatic aberration and further enhance a patient's range of vision while minimizing side effects. A Kinoform lens structure may further provide for enhanced focusing efficiency, e.g. a Kinoform made out of a non-absorbing but refracting material may have 100% focusing efficiency, further enhancing a patient's range of vision while minimizing side effects. A diffractive structure may be attached to, placed on or formed onto a refractive element's posterior or anterior surface. A diffractive structure may have a back surface being congruent with a refractive element's posterior or anterior surface or it may be smaller, e.g. limited to the central-lens area, e.g. 3 mm to 4 mm in diameter. The latter may further allow improved mesopic and scotopic vision.

A Fresnel structure may be or comprise a Kinoform lens structure. Kinoform lenses may be diffractive/refractive optics taking advantage of both modalities which may ensure maximum efficiency. A Kinoform lens structure may have a (general) parabolic surface profile (e.g. a surface-relief profile). A Kinoform lens structure may exhibit zones (e.g. extending from a minimum to a maximum height in the structure) and step heights (i.e. the maximum height difference within a zone). The zones in a Kinoform lens structure may exhibit the form of circular annular rings; however the zones may assume other shapes, such as bars (as in a conventional spectrographic grating) and ellipses (to produce different focal lengths at different angular orientations about the optical axis). Moreover, groups of zones may be arranged in a two-dimensional cellular array, with each cell functioning as an individual optical element. Within each zone, the theoretically optimal depth profile may be a smooth curve extending continuously from a highest region to a lowest region; however for ease of manufacturability, the optimal profile may be approximated as a series of steps (phase levels) each of constant depth. A surface-relief profile t(r) may be determined approximately from the relation t(r)=[λ/2π(μ−1)][Φ(r)]2π, where λ is the light wavelength, μ is the refractive index of the optical material (of the surface-relief profile), and [Φ(r)]2π is the phase function Φ(r) modulo 2π. A Kinoform lens structure may exhibit an arbitrary phase profile, for example the interference pattern defined by two arbitrary coherent point sources on an arbitrary surface, to which an arbitrary 2-dimensional phase profile defined by an arbitrary polynomial may be added. In particular, a Kinoform lens structure may be described by way of the interference pattern produced on a flat plane normal to the optical axis defined by the two points, with one point at infinity. A Kinoform lens structure may be produced by etching or machining ridges into a surface, e.g. a (posterior and/or anterior) surface of a/the refractive element, e.g. by step approximations, or gray-scale direct milling IBL. Kinoform lens structures may be made from an optical material, e.g. an amorphous or single crystalline material, such as PMMA, silicone, acrylate. For instance, Kinoform lens structures may comprise or be made from a plastic e.g. polymethyl methacrylate (PMMA), hydrophobic acrylic (e.g., phenylethyl methacrylate (PEMA) and phenylethyl acrylate (PEA)); hydrophilic acrylic (e.g., poly hydroxyethylmethacrylate (pHEMA)); and/or silicone (e.g., poly dimethylsiloxane (PDMS)).

A Kinoform lens structure may be composed of or comprise a continuous “sawtooth” surface profile that may induce ray divergence as may be further detailed below by way of examples. In particular the (negative) Kinoform lens structure may exhibit a diffractive (surface) profile (which may be at least partly superimposed on a surface of the refractive element) having a local minimum in the centre, so for example having no outward pointing (with respect to the refractive element, e.g. along the optical axis) central bulge, and the distance between the “sawtooth” maxima may decrease with distance to the centre. For instance, the Kinoform lens structure may have a central zone lower with respect to a first diffractive step (first w.r.t. the centre).

A Kinoform lens structure may be or comprise a Fresnel phase zone plate structure. A Fresnel phase zone plate structure may be similar in appearance to a Fresnel lens structure. A Fresnel phase zone plate structure may comprise several concentric rings (corresponding to respective zones) having a zone radius. From the side a Fresnel phase zone plate structure may look like a series of sawtooth-shaped ridges. A Fresnel phase zone plate structure may be based on diffraction rather than refraction and may have many more zones (e.g. 10 to 40 or 15 to 30) and a much smaller difference in height (i.e. step height) between adjacent zones compared to a Fresnel lens. The step height within a zone may e.g. correspond to a phase change of 2π (one wavelength). The step height may e.g. be at least 0.1 μm, at least 0.6 μm or at least 1 μm and/or at most 10 μm, e.g. the step height may be 6.7 μm. The Fresnel phase zone plate structure may e.g. comprise 10 to 40 or 15 to 30 zones (e.g. in form of concentric rings around the optical axis of a/the refractive element). The minimum zone radius may be at least 0.4 mm, in particular at least 0.55 mm or at least 0.76 mm, and/or at most 1 mm. For instance, LCA may be dependent on materials and (optical) powers of the implantable lens and/or refractive element and/or element inducing an increase of the chromatic aberration. An increase of LCA may be an increase by a certain value (e.g. from a base power of 0.25 to 3 or 4D). For an implantable lens having a refractive element with a base power between zero and 40D, a unique Kinoform (lens) structure may to be designed. For instance, a Kinoform lens structure may be formed onto or into a posterior and/or anterior surface of a refractive element. The Kinoform lens structure may extend from the centre of a refractive element to its edge, e.g. from the centre of the posterior or anterior surface to the edge of the posterior or anterior surface, respectively. Alternatively, the Kinoform lens structure may extend from the centre of a refractive element to a distance smaller than the distance between the centre of the refractive element and its' edge, e.g. from the centre of the posterior or anterior surface to a (radial) distance smaller than the maximum (radial) distance from the centre of the posterior or anterior surface to the edge of the (respective) posterior or anterior surface, e.g. a (radial) distance of 1.5 to 2 mm (e.g. 2.25 mm) (measured from the centre). The Kinoform lens structure may, hence, e.g. be limited to a central area of the refractive element's surface, e.g. 3 mm to 4 mm/4.5 mm in diameter, which may further allow improved mesopic and scotopic vision.

According to a further example embodiment of the implantable lens according to the first example aspect the refractive element has a posterior and an anterior surface and the diffractive structure is on the posterior and/or the anterior surface. In this way the diffractive structure can be advantageously placed and/or distributed on the surface of the refractive element and may, hence, further enhance a patient's range of vision while minimizing side effects.

According to a further example embodiment of the implantable lens according to the first example aspect the diffractive structure is a negative diffractive structure, in particular having a power of at most −1D, preferably the power is between −1D and −11D (i.e. at least −11 D and/or at most −1 D) and more preferably at least-8 D and/or at most −3 D, e.g. −7 D. A negative diffractive structure, in particular having a power of at most −1D, may further enhance a patient's range of vision while minimizing side effects. A power of less than −11D may, however, not be cost-effectively manufactured and may produce further side effects.

According to a further example embodiment of the implantable lens according to the first example aspect the diffractive structure comprises a diffractive profile and in particular the diffractive profile extends from a first Fresnel zone of the diffractive structure to an edge of the refractive element or the diffractive profile extends from the centre of the refractive element to an edge of the refractive element. A diffractive profile may be a profile that causes diffraction of light. A diffractive profile may be a profile formed, e.g, etched or laser formed, into a posterior and/or anterior surface. The first Fresnel zone may e.g. be centred in the centre of a posterior or anterior surface, e.g. the axis about which a first Fresnel zone is rotational symmetric may be the optical axis of the refractive element. The edge may e.g. be the edge of the anterior or posterior surface. Using a diffractive profile, e.g. a Kinoform profile (i.e. a diffractive profile having a Kinoform lens structure) further enhance a patient's range of vision while minimizing side effects.

The diffractive profile may be a continuous “sawtooth” surface profile that may have a local minimum in the centre, so for example having no outward pointing central bulge, and the distance between the “sawtooth” maxima may decrease with distance to the centre.

According to a further example embodiment of the implantable lens according to the first example aspect the refractive element and the element inducing an increase of the chromatic aberration together comprise or form a diffractive-refractive zone (approximately) centred on the centre of the refractive element, in particular the diffractive-refractive zone has a diameter from 3 mm to 4.5 mm (and for instance a surface of the refractive element may have a diameter of 5.5 to 7 mm on which the diffractive-refractive zone is formed). The diffractive-refractive zone may e.g. be formed by a diffractive profile, e.g. together by the refractive element (e.g. the surface having a concave or convex form) and a diffractive profile formed, e.g, etched, into the surface of the refractive element. The diffractive-refractive zone may e.g. be formed by a Kinoform profile. The centre of the refractive element may be defined by the optical axis of the implantable lens. An optical axis may be an axis around which the implantable lens and/or refractive element exhibits (approximately) rotational symmetry. An optical axis may e.g. pass through the centre of curvature of each surface of the implantable lens and/or the refractive element, and may coincide with an axis of rotational symmetry. The optical axis may e.g. coincide with the mechanical axis of the implantable lens and/or refractive element. A diffractive-refractive zone may allow further enhancing a patient's range of vision while minimizing side effects.

A/the part of the refractive element's surface not covered by a diffractive-refractive zone, e.g. an outer zone, may be only refractive, without induced diffractive effects. An outer zone may be a ring with an inner diameter of 3 to 4.5 mm and an outer diameter of 5.5 to 7 mm or extending to the edge of the respective surface of the refractive element. An outer zone may allow for a refractive power compensation and may further enhance a patient's range of vision while minimizing side effects.

According to a further example embodiment of the implantable lens according to the first example aspect the diffractive structure utilizes the first and/or second (e.g. positive or negative) diffractive order. For instance, a simultaneous action of both first and second orders may be utilized, e.g. in case the implantable lens and/or refractive element are or comprise a bifocal or trifocal lens, e.g. in which first and second order are used simultaneously. This may allow for a further enhancement of a patient's range of vision while minimizing side effects.

According to a further example embodiment of the implantable lens according to the first example aspect the diffractive element and the element inducing an increase of the chromatic aberration direct light to the same focus with respect to a certain light wavelength, e.g. a wavelength of 550 nm, 555 nm or 546 nm. This may allow for a further enhancement of a patient's range of vision while minimizing side effects.

According to a further example embodiment of the implantable lens according to the first example aspect the implantable lens has a monochromatic modulation transfer function (MTF) at 100 lp/mm of 0.43 or better (e.g. higher), preferably a monochromatic modulation transfer function (MTF) at 100 lp/mm of 0.5 or better (e.g. higher), more preferably 0.6 or better. The implantable lens may meet optical-quality criteria for a monofocal lens as e.g. defined in the standard ISO 11979-2:2014. Such an implantable lens may allow for enhanced optical performance. This may allow for a further enhancement of a patient's range of vision while minimizing side effects.

According to a further example embodiment of the implantable lens according to the first example aspect the refractive element has a nominal power range of −5 D to 40 D or 0 D to 40 D, in particular 20 D to 30 D. This may allow for a further enhancement of a patient's range of vision while minimizing side effects.

According to a further example embodiment of the implantable lens according to the first example aspect the element inducing an increase of the chromatic aberration increases the chromatic aberration by at least 1D, preferably at least 2D, at a spectral range from 450 nm to 650 nm. For instance, the element inducing an increase of the chromatic aberration may increase a pseudophakic eye's chromatic aberration by at least 1D, preferably at least 2D, at a spectral range from 450 nm to 650 nm. This may allow for a further enhancement of a patient's range of vision while avoiding or minimizing side effects.

According to a further example embodiment of the implantable lens according to the first example aspect the element inducing an increase of the chromatic aberration and the refractive element consist of the same material, in particular the element inducing an increase of the chromatic aberration and the refractive element form an integral unit, e.g. by the element inducing an increase of the chromatic aberration being formed on or in the refractive element. This may allow for a further enhancement of a patient's range of vision while minimizing side effects.

According to a further example embodiment of the implantable lens according to the first example aspect, the Kinoform lens structure comprises a sawtooth surface profile, in particular superimposed on an anterior and/or posterior surface of the refractive element, wherein optionally the Kinoform lens structure may have a central zone lower with respect to a first diffractive step.

According to a further example of the implantable lens according to the first example aspect, the sawtooth surface profile exhibits a local minimum in the centre (i.e. at radius 0), in particular the distance between the maxima, where the sawtooth surface profile reaches the step height, decreases with distance to the centre. The centre may correspond to the centre of the refractive element, for instance when the sawtooth surface profile is centred in the centre of the refractive element. To account for any irregularities induced by the surface structure of the refractive element (e.g. a convex surface), maxima and minima may be determined against a baseline, which corresponds to the respective surface of the refractive element.

According to a second example aspect, above object is solved by a method for producing an implantable lens according to the first example aspect, the method comprising etching and/or machining an element inducing an increase of the chromatic aberration on an anterior and/or a posterior surface of a refractive element.

According to a further example embodiment of the implantable lens according to the first example aspect may be a hybrid refractive-diffractive intraocular lens. For instance, the implantable lens may have a posterior and anterior surface one or both being e.g. aspheric or having e.g. a spherical shape and one or both having e.g. a Fresnel or Kinoform lens structure yielding a refractive effect of a negative (diverging) lens to e.g. compensate a surplus of positive refractive power of the base refractive element whereas both diffractive and refractive structure contribute to a single focus. The implantable lens may comprise or consist of a refractive element having refractive dispersion and a Fresnel or Kinoform lens structure having diffractive dispersion. The (Fresnel) lens power of a Fresnel or Kinoform lens structure e.g. ranging from −1D to −11D. A (hybrid) refractive-diffractive intraocular lens may have a nominal power range of abut OD to about 40 D. The implantable lens may be designed to increase a pseudophakic eye's chromatic aberration by about 1D at a spectral range from 450 nm to 650 nm e.g. by combining a refractive and diffractive chromatic aberration. The intraocular lens described in claim 2 that increases a pseudophakic eye's chromatic aberration by about 2D at a spectral range from 450 nm to 650 nm by combining a refractive and diffractive chromatic aberration. The implantable lens, e.g. a refractive-diffractive intraocular lens, may e.g. utilize the first (e.g. positive or negative) diffractive order of the negative diffractive element and/or the second (e.g. positive or negative) diffractive order of the negative diffractive element. The implantable lens, e.g. a refractive-diffractive intraocular lens, may have a diffractive profile spanning from a first Fresnel zone to the lens' edge. The implantable lens, e.g. a refractive-diffractive intraocular lens, may have a diffractive-refractive zone in the lens centre with the zone diameter ranging from 3 mm to 4.5 mm and a purely refractive outer zone with a refractive power compensation. The implantable lens, e.g. a refractive-diffractive intraocular lens, may have a monochromatic MTF at 100 lp/mm of 0.43 or better.

The disclosed example aspects and example embodiments may allow for providing an extended depth of focus that addresses the needs of pseudophakic patient, e.g. after cataract surgery, in particular with respect to enhancing a patient's range of vision with minimal or no side effects.

The features and example embodiments described above may equally pertain to the different aspects.

### BRIEF DESCRIPTION OF THE DRAWINGS

Other features and advantages will become apparent from the following detailed description considered in conjunction with the accompanying drawings.

FIG. 1 shows an example embodiment of an implantable lens according to the first example aspect;

FIG. 2 shows an example embodiment of an IOL not according to the first example aspect;

FIG. 3 shows an example embodiment of an implantable lens according to the first example aspect;

FIG. 4a shows an example diffractive profile of an element inducing an increase of the chromatic aberration, in particular of a Kinoform lens structure, of an implantable lens according to the first example aspect;

FIG. 4b shows an example phase map (Modulo-2π) of an example diffractive surface of an element inducing an increase of the chromatic aberration, in particular a phase map of an example Kinoform lens structure, of an implantable lens according to the first example aspect;

FIG. 5a shows an example modulation transfer function of the example embodiment of FIG. 4a/4b;

FIG. 5b shows an example modulation transfer function of the example embodiment of FIG. 4a/4b;

FIG. 6a shows an example diffractive profile of an element inducing an increase of the chromatic aberration, in particular of a Kinoform lens structure, of an implantable lens according to the first example aspect;

FIG. 6b shows an example phase map (Modulo-2π) of an example diffractive surface of an element inducing an increase of the chromatic aberration, in particular a phase map of an example Kinoform lens structure, of an implantable lens according to the first example aspect;

FIG. 7a shows an example modulation transfer function of the example embodiment of FIG. 6a/6b;

FIG. 7b shows an example modulation transfer function of the example embodiment of FIG. 6a/6b;

FIG. 8 shows an example phase map (Modulo-2π) of an example diffractive surface of an element inducing an increase of the chromatic aberration, in particular a phase map of an example Kinoform lens structure, of an implantable lens according to the first example aspect;

FIG. 9a shows an example modulation transfer function of the example embodiment of FIG. 8;

FIG. 9b shows an example modulation transfer function of the example embodiment of FIG. 8;

FIG. 10a shows a retinal-image simulations obtained in an ISO 11979-2 model eye with an implantable lens according to the first example aspect;

FIG. 10b shows a retinal-image simulations obtained in an ISO 11979-2 model eye with an implantable lens according to the first example aspect;

FIG. 10c shows a retinal-image simulations obtained in an ISO 11979-2 model eye with an implantable lens according to the first example aspect;

FIG. 11 shows an example logMAR visual acuity;

FIG. 12a illustrates an example embodiments of an implantable lens; and

FIG. 12b illustrates an example embodiments of an implantable lens.

### DETAILED DESCRIPTION

The following description serves to deepen the understanding and shall be understood to complement and be read together with the description as provided in the above summary section of this specification. Some aspects may have a different terminology than e.g. provided in the description above. The skilled person will nevertheless understand that those terms refer to the same subject-matter, e.g. by being more specific. For instance, a diffractive structure may be referred to as a diffractive grating; a base (refractive) power may be referred to as a (nominal) power.

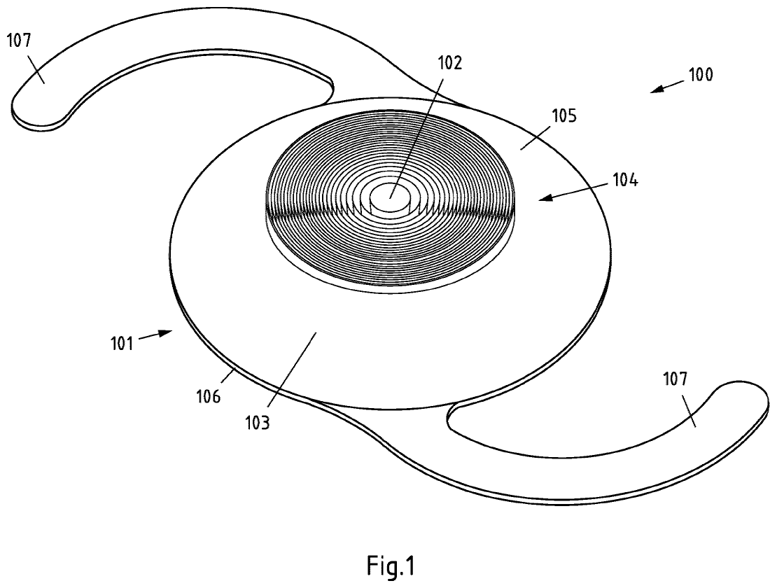

FIG. 1 shows an example implantable lens 100 according to the first example aspect comprising a refractive element 101 causing chromatic aberration and an element 102 inducing an increase of the chromatic aberration. The element 102 inducing the increase of the chromatic aberration comprises a diffractive structure 102, in particular a Kinoform lens structure 102. The refractive element 101 has an anterior surface 103 and the diffractive structure 102 is (centred) on the anterior surface 103, the axis about which the Kinoform lens structure is rotational symmetric is the optical axis of the refractive element 101. The refractive element 101 and the element 102 inducing an increase of the chromatic aberration together comprise a diffractive-refractive zone 104 centred on the centre of the refractive element 101. The diffractive structure 102 is a negative diffractive structure 102. The part of the refractive element's 101 surface 103 not covered by a diffractive-refractive zone 104, is the outer zone 105, which may e.g. be only refractive without induced refractive effects and/or without diffractive effects. Alternatively, the diffractive structure 102 comprises a diffractive profile extending from a first Fresnel zone of the diffractive structure to an edge 106 of the refractive element or the diffractive profile extends from the centre of the refractive element to an edge 106 of the refractive element. The implantable lens 100 may be an IOL 100, the refractive element 101 may be (e.g. plastic) lens 101 with (e.g. plastic) side struts 107, e.g. haptics 107, to hold the lens in place in a capsular bag inside an eye.

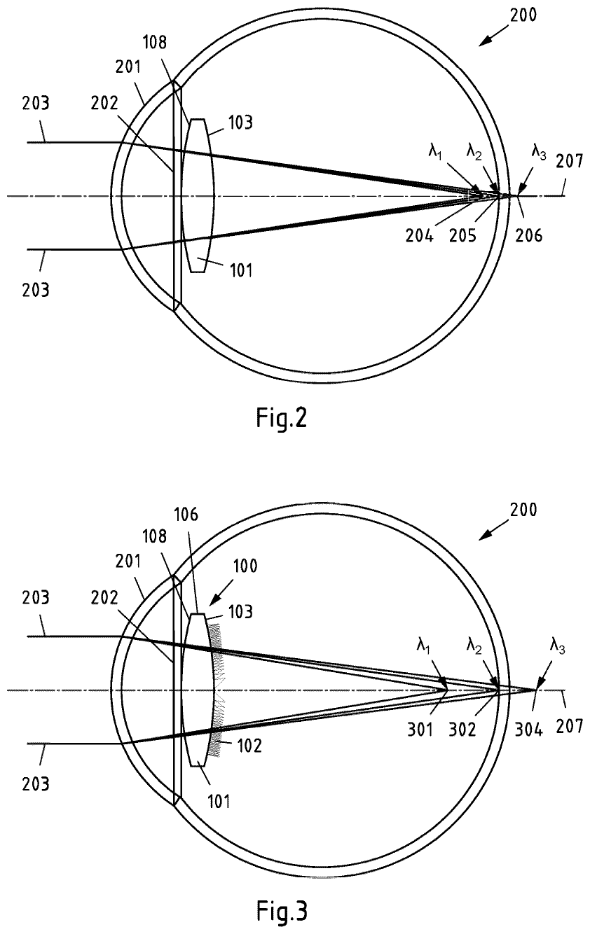

FIG. 2 shows a case in which an IOL not according to the first example aspect is implanted within an eye 200 having a refractive element 101 causing chromatic aberration. Due to the chromatic aberration caused by the refractive element 101 light 203 of different wavelength lambda_1, lambda_2 and lambda 3 is directed to different foci or focal points 204, 205, 206, for instance blue light 203 of wavelength lambda_1, e.g. 450 nm, may be directed to focus 204, green light 203 of wavelength lambda_2, e.g. 550 nm, may be directed to focus 205, and red light 203 of wavelength lambda_3, e.g. 650 nm, may be directed to focus 206. Optical axis 207 coincides with axis of rotational symmetry of refractive element 101.

FIG. 3 shows an example implantable lens 100 according to the first example aspect comprising a refractive element 101 (which may be the refractive element shown in FIG. 2) causing chromatic aberration and an element 102 inducing an increase of the chromatic aberration. The element 102 inducing the increase of the chromatic aberration comprises a diffractive structure 102, in particular a Kinoform lens structure 102. The refractive element 101 has an anterior 108 and posterior surface 103, wherein the diffractive structure 102 is on the posterior surface 103 and centred on the centre of the posterior surface 103, i.e. centred on the posterior surface 103, such that the axis 207 about which the diffractive structure 102, in particular the Kinoform lens structure 102, is rotational symmetric coincides with the optical axis 207 of the refractive element 101. The diffractive structure 102 may be a negative diffractive structure 102. The element 102 inducing an increase of the chromatic aberration, e.g. the diffractive structure 102 may cover (almost) the entire posterior surface 103, e.g. the structure may extend from the centre of the refractive element 101 to the edge 106 of the refractive element 101. The example implantable lens 100 is implanted within an eye 200. Due to the chromatic aberration light 203 of different wavelength lambda_1, lambda_2 and lambda 3 is directed to different foci or focal points 301, 302, 304, for instance blue light 203 of wavelength lambda_1, e.g. 450 nm, may be directed to focus 301 and red light 203 of wavelength lambda_3, e.g. 650 nm, may be directed to focus 304. Optical axis 207 coincides with axis of rotational symmetry of refractive element 101. The implantable lens 100 is, in this example, implanted in a capsular bag inside an eye 200 behind the cornea 201 and iris 202. The refractive element and the element inducing an increase of the chromatic aberration direct light to the same focus with respect to a certain light wavelength e.g., with respect to a design wavelength, e.g. 550 nm. An element 102 induces an increase of the chromatic aberration with respect to the (intrinsic) chromatic aberration of the refractive element 101. For instance, the focus for a design wavelength lambda_2, e.g. green light, e.g. 550, 555, or 456 nm, of the refractive element 101 may be the same as for the refractive element 101 alone (compare to FIG. 2), while the focus for blue light having a wavelength lambda_1 of 450 nm may shift (with respect to the focus for lambda_3 wavelength for the refractive element 101 alone) closer to the refractive element 101, whereas the focus for red light having a wavelength lambda_3 of 650 nm may shift away from the refractive element due to the element 102 inducing an increase of the chromatic aberration, such that the distance between the focus for red light and for blue light is increased with respect to the distance between the focus for red light and for blue light of the refractive element 101 (alone, without the element 102 inducing an increase in chromatic aberration).

An implantable lens, e.g. an ophthalmic lens, e.g., IOL, according to the first example aspect may induce an excess of longitudinal chromatic aberration (LCA) to extend the depth of focus (DoF) of an eye after e.g. crystalline lens removal and (IOL) implantation. Embodiments of the implantable lens according to the first example aspect may increase LCA through a refractive and diffractive principle and may be applied in monofocal IOLs, e.g. in standard monofocal IOLs. The application may, however, also be extended to multifocal IOLs to counter visual-quality gaps observed between designed foci seen in contemporary technology. The implantable lens according to the first example aspect may be applicable to standard capsular-bag implants as well as supplementary or phakic IOLs.

Embodiments of the implantable lens according to the first example aspect may e.g. be used to correct aphakia after cataract surgery or refractive lens exchange to provide good distance vision and expand the eye's DoF. Such embodiments may e.g. be hybrid lenses with a base (refractive) power, e.g. of the refractive element, and a diffractive grating, e.g. of an element inducing an increase of the chromatic aberration, disposed on their front, back, or both surfaces.

In one example, a refractive element (e.g. a (biconvex) lens) has a pattern of diffractive grooves (e.g. a Kinoform lens structure) located posteriorly, e.g. on a posterior surface of the refractive element or lens. Such a diffractive grating may have a refractive effect of a negative lens; thus, the refractive base power (e.g. of the refractive element) may be chosen higher than the nominal power to compensate for the power reduction. This example embodiment of the implantable lens according to the first aspect may increase the LCA while maintaining the position of the blue focus more anteriorly with respect to the central wavelength (i.e. design wavelength, e.g. 550 nm) as compared to the red focus placed more posteriorly, which may make it comparable to the natural condition. However, other example embodiments with lower base power and positive refractive power of the grating that induces (an excess of) negative LCA can also be used.

In a further example embodiment of an implantable lens according to the first example aspect, the example refractive element has a posterior and anterior surface, the posterior and the anterior surface having a spherical shape, with the refractive element resembling a biconvex lens. The example refractive element has an optical power or nominal power of 20 D. The example element inducing the increase of the chromatic aberration is an example Kinoform lens structure (formed) on the posterior surface of the example refractive element, the example Kinoform lens structure e.g. having a central thickness of 1 mm. The example Kinoform lens structure has (e.g. consists of) 15 zones. The example refractive element and the example Kinoform lens structure together form an example diffractive-refractive zone centred on the centre of the example refractive element. The example element inducing an increase of the chromatic aberration and the refractive element consist of the same example material and form an integral unit, the example material having an Abbe number of 46 and a refractive index of 1.50.

For example, an implantable lens was constructed using a material having a refractive index of 1.50 and an Abbe number of 46. A central lens thickness was assumed to be 1 mm, and a nominal power of +20 D was set. A model eye may be built in ZEMAX Optic Studio (by Radiant Zemax LLC) in accordance with ISO 11979 to test the optical quality and defocus tolerance of the proposed embodiments. In case of such a test, optical simulations may be performed in polychromatic light with the spectral weighting corresponding to the CIE photopic luminosity function at the range of 450 to 650 nm.

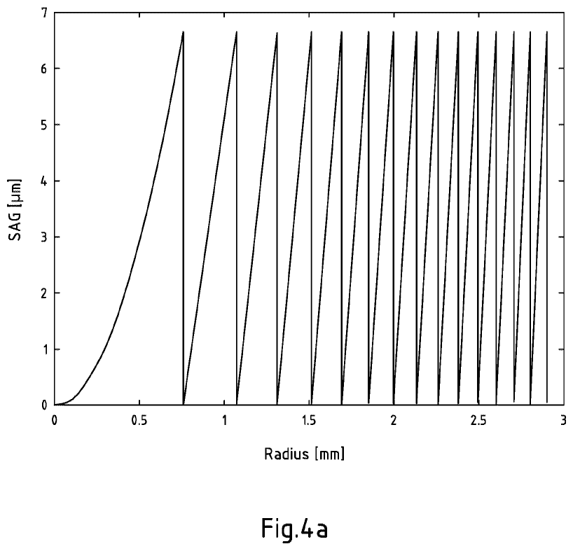

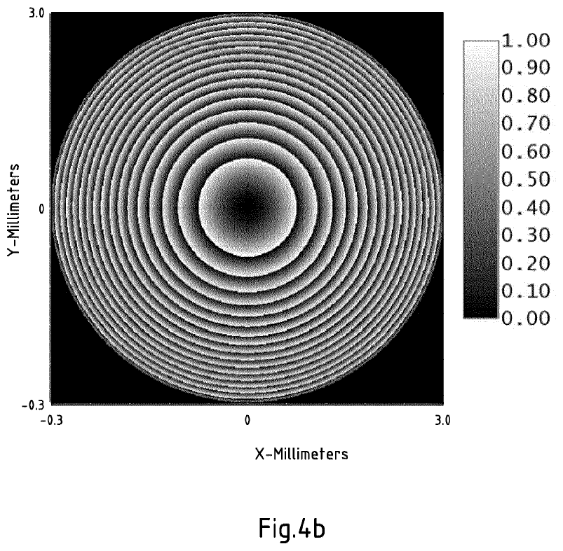

In a further example embodiment of an implantable lens according to the first example aspect, the example refractive element has a posterior and anterior surface, the posterior and the anterior surface having a spherical shape, with the refractive element resembling a biconvex lens. The example refractive element has a base (optical) power or nominal power of 23.75 D and e.g. having a central thickness of 1 mm. The example element inducing the increase of the chromatic aberration is an example Kinoform lens structure (an example (radial) profile of which is depicted in FIGS. 4a/4b) (formed) on the posterior surface of the example refractive element, the example Kinoform lens structure having a (optical) power of −3.75 D. The example Kinoform lens structure has (e.g. consists of) 15 zones having a minimal zone radius of 0.76 mm (cf. FIG. 4a) and a step height of 6.7 μm. The example refractive element and the example Kinoform lens structure together comprise an example diffractive-refractive zone centred on the centre of the example refractive element. The example Kinoform lens structure utilizes the second diffractive order (m=2). The example element inducing an increase of the chromatic aberration and the refractive element consist of the same example material and form an integral unit, e.g. the example material having an Abbe number of 46 and a refractive index of 1.50. The example element inducing an increase of the chromatic aberration, wherein the example increase of the chromatic aberration is an increase by a factor of two (over the chromatic aberration caused by the refractive element and the eye). The distance between foci between light of 486 and 656 nm may be increased to 5654 mm, when measured in an immersion with 1.336 refractive index of the surrounding medium, and 880 mm, when measured in the eye/in situ. The refractive element (without the element increasing the chromatic aberration) may cause the distance between foci between light of 486 and 656 nm to be 1364 mm, when measured in an immersion with 1.336 refractive index of the surrounding medium, and 479 mm, when measured in the eye/in situ.

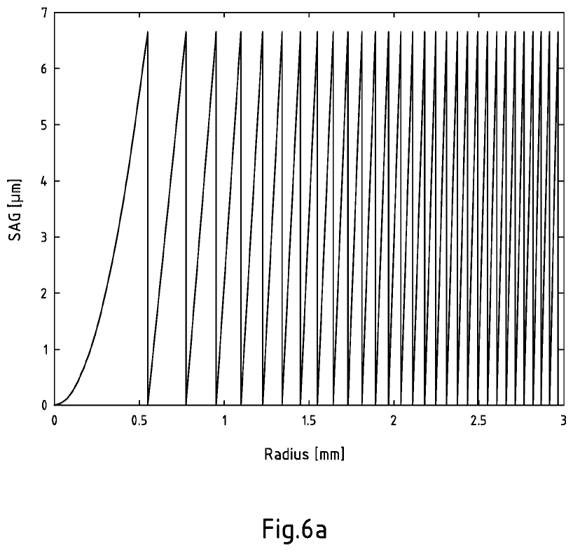

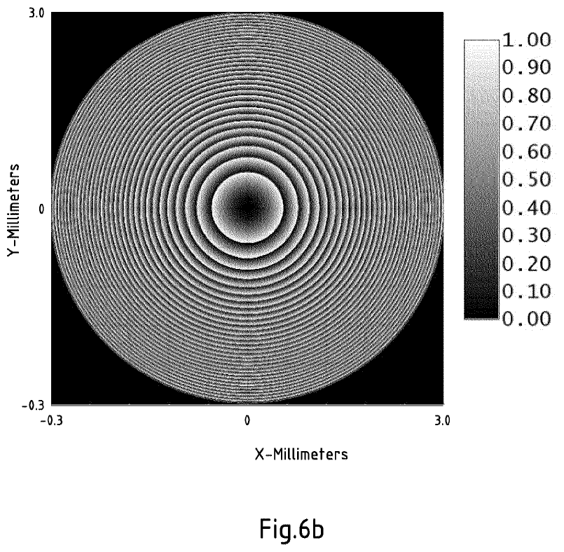

For example, a positive refractive base with a power of 23.75 D was combined with a negative Fresnel lens (−3.75 D), which effectively doubled the pseudophakic eye's chromatic aberration. The second diffractive order (m=2) is used, and the diffractive surface consists of 15 Fresnel zone plates with a minimum zone radius of 0.76 mm (cf. FIG. 6a). For the selection of m=2, the step height was 6.7 μm which may ensure the maximum efficiency. If, however, a different (e.g. positive or negative) diffractive order is to be used, the step height may be adjusted accordingly. For instance, for the selection of m=1, the first Fresnel zone radius may be 0.54 mm, and the step height may be 0.003 or 0.0033 mm. The example embodiment depicts a full-diffractive design with grating disposed on the back surface. Other embodiments that have the grating limited to the central-lens area (e.g., 3 mm or 4 mm) may allow for improved mesopic and scotopic vision. The example embodiment may be constructed using a material having a refractive index of 1.50 and an Abbe number of 46. A central lens thickness was e.g. assumed to be 1 mm. The chromatic focus shift between 486 nm and 656 nm may be 5654 μm measured in immersion (1.336 refractive index of the surrounding medium). The chromatic focus shift between 486 nm and 656 nm may be 880 μm measured in the eye/in situ. For the lens without additional LCA, the chromatic shift may be 1364 μm (immersion) and 479 μm (in situ).

FIG. 4a shows an example diffractive profile of an element inducing an increase of the chromatic aberration, in particular of a Kinoform lens structure, of an implantable lens according to the first example aspect, further particulars may be given by the example above. The step height was 6.7 μm (cf. the ordinate showing sagitta (SAG)). The diffractive profile utilizes a 2-fold increase of the eye's longitudinal chromatic aberration. The diffractive profile is a (continuous) sawtooth surface profile having a local minimum in the centre (at radius 0 mm). The baseline of the diffractive profile may follow a surface of the refractive element, so it may be superimposed on a (posterior or anterior) surface of the refractive element. Further, the distance between the “sawtooth” maxima, where the profile reaches the step height, decreases with distance to the centre. FIG. 4b shows an example phase map (Modulo-2π) of this example embodiment.

For instance, the element inducing an increase of the chromatic aberration may be an element inducing ray divergence and the example diffractive profile shown in FIG. 4a may be of an element inducing ray divergence and having a negative focal length, in particular of a Kinoform lens structure with a central zone lower with respect to the first diffractive step, of an implantable lens according to the first example aspect.

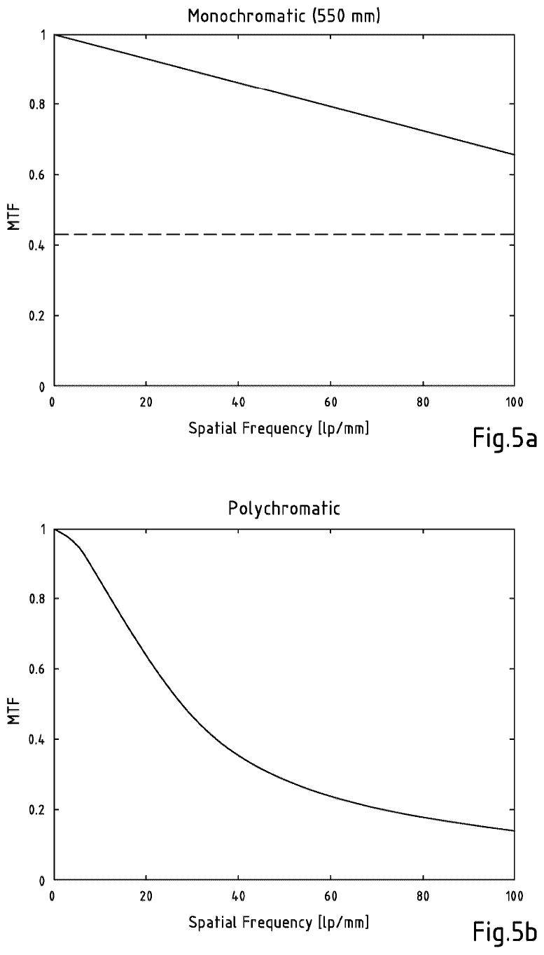

The simulated optical quality in the model eye meets the requirements of the manufacturing standards (ISO 11979-2) for monofocal lenses requiring a monochromatic (550 nm) modulation transfer function (MTF) at 100 lp/mm greater than or equal to 0.43. The described example embodiment exceeds this requirement with an MTF value of 0.66, indicating a nearly diffraction-limited performance (FIG. 5a). FIG. 5b shows a polychromatic MTF of the discussed embodiment.

FIG. 5a and FIG. 5b show modulation transfer function (MTF) levels of one example embodiment with a 2-fold increase of longitudinal chromatic aberration in a model eye. Monochromatic (FIG. 5a) and polychromatic (FIG. 5b) MTFs (solid lines) are shown up to 100 lp/mm. The dashed line (FIG. 5a) indicates the lowest (monochromatic) MTF at 100 lp/mm accepted for monofocal IOLs.

In a further example embodiment of an implantable lens according to the first example aspect, the example refractive element has a posterior and anterior surface, the posterior and the anterior surface having a spherical shape, with the refractive element resembling a biconvex lens. The example refractive element has a base (optical) power or nominal power of 27.25 D. The example element inducing the increase of the chromatic aberration is an example Kinoform lens (an example (radial) profile of which is depicted in FIG. 6a/6b) (formed) on the posterior surface of the example refractive element, the example Kinoform lens structure having a (optical) power of −7.25 D. The example Kinoform lens structure has (e.g. consists of) 30 zones having a minimal zone radius of 0.55 mm (cf. FIG. 6a) and a step height of 6.7 μm. The example refractive element and the example Kinoform lens structure together form an example diffractive-refractive zone centred on the centre of the example refractive element. The example Kinoform lens structure utilizes the second diffractive order. The example element inducing an increase of the chromatic aberration and the refractive element consist of the same example material and form an integral unit, the example material having an Abbe number of 46 and a refractive index of 1.50. The example element inducing an increase of the chromatic aberration, wherein the example increase of the chromatic aberration is an increase by a factor of three (over the chromatic aberration caused by the refractive element and the eye). The distance between foci between light of 486 and 656 nm may be increased to 9722 mm, when measured in an immersion with 1.336 refractive index of the surrounding medium, and 1247 mm, when measured in the eye/in situ. The refractive element (without the element increasing the chromatic aberration) may cause the distance between foci between light of 486 and 656 nm to be 1364 mm, when measured in an immersion with 1.336 refractive index of the surrounding medium, and 479 mm, when measured in the eye/in situ.

For example, a positive refractive base with a power of 27.25 D was combined with a negative Fresnel lens (−7.25 D), which effectively tripled the pseudophakic eye's chromatic aberration. The second diffractive order (m=2) is used, the diffractive surface consists of 30 Fresnel zone plates with a minimum zone radius of 0.55 mm (FIG. 3). For the selection of m=2, the step height was 6.7 μm which may ensure the maximum efficiency. If, however, a different (e.g. positive or negative) diffractive order is to be used, the step height may be adjusted accordingly. For instance, for the selection of m=1, the first Fresnel zone radius may be 0.39 mm, and the step height may be 0.003 or 0.0033 mm. This example embodiment depicts a full-diffractive design with grating disposed on the back surface. Other embodiments that have the grating limited to the central-lens area (e.g., 3 mm or 4 mm) may allow for improved mesopic and scotopic vision. The example embodiment may be constructed using a material having a refractive index of 1.50 and an Abbe number of 46. A central lens thickness was e.g. assumed to be 1 mm. The chromatic focus shift between 486 nm and 656 nm may be 9722 μm measured in immersion (1.336 refractive index of the surrounding medium). The chromatic focus shift between 486 nm and 656 nm may be 1247 μm measured in the eye/in situ. For the lens without additional LCA, the chromatic shift may be 1364 μm (immersion) and 479 μm (in situ).

FIG. 6a shows an example diffractive profile of an element inducing an increase of the chromatic aberration, in particular of a Kinoform lens structure, of an implantable lens according to the first example aspect, further particulars may be given by the example above. The diffractive profile utilizes a 3-fold increase of the eye's longitudinal chromatic aberration. The diffractive profile is a continuous “sawtooth” surface profile having a local minimum in the centre (at radius 0 mm). The baseline of the diffractive profile follows a surface of the refractive element, so it is superimposed on a (posterior or anterior) surface of the refractive element. Further, the “sawtooth” maxima, where the profile reaches the step height, decrease with distance to the centre. FIG. 6b shows an example phase map (Modulo-2π) of this example embodiment.

For instance, the element inducing an increase of the chromatic aberration may be an element inducing ray divergence and the example diffractive profile shown in FIG. 6a may be of an element inducing ray divergence and having a negative focal length, in particular of a Kinoform lens structure with a central zone lower with respect to the first diffractive step, of an implantable lens according to the first example aspect.

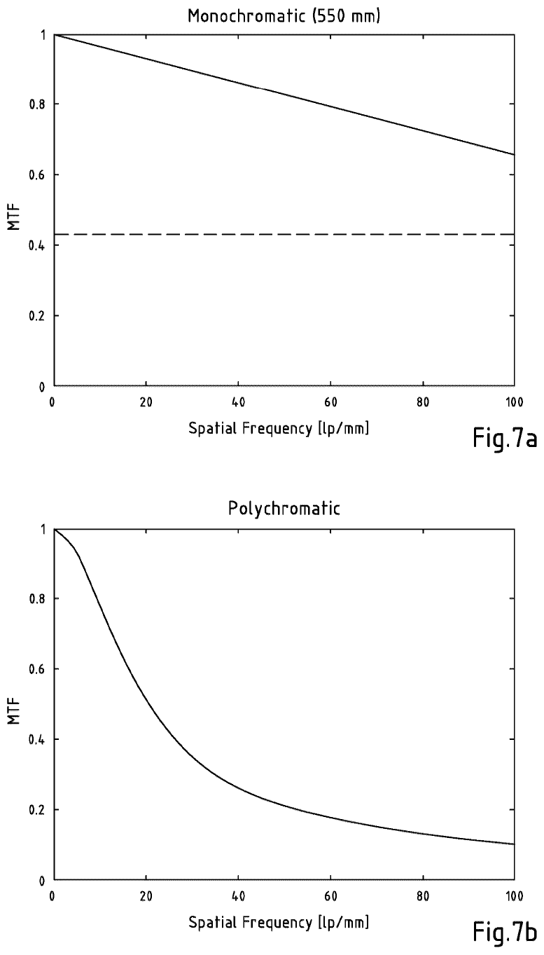

The simulated optical quality in the model eye meets the requirements of the manufacturing standards (ISO 11979-2) for monofocal lenses requiring a monochromatic (550 nm) modulation transfer function (MTF) at 100 lp/mm greater than or equal to 0.43. The described example embodiment exceeds this requirement with an MTF value of 0.65, indicating a nearly diffraction-limited performance (FIG. 7a). FIG. 7b shows a polychromatic MTF of the discussed embodiment.

FIG. 7a and FIG. 7b show modulation transfer function (MTF) levels of one example embodiment with a 3-fold increase of longitudinal chromatic aberration in a model eye. Monochromatic (FIG. 7a) and polychromatic (FIG. 7b) MTFs (solid lines) are shown up to 100 lp/mm. The dashed line (FIG. 7a) indicates the lowest (monochromatic) MTF at 100 lp/mm accepted for monofocal IOLs.

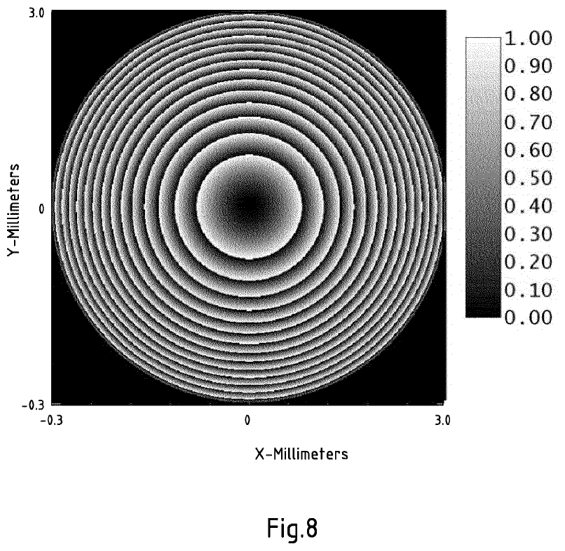

In a further example embodiment of an implantable lens according to the first example aspect, the example refractive element has a posterior and anterior surface, the posterior and the anterior surface having a spherical shape, with the refractive element resembling a biconvex lens. The example refractive element has a base (optical) power or nominal power of 23.5 D. The example element inducing the increase of the chromatic aberration is an example Kinoform lens structure (cf. the phase map (Modulo-2π) in FIG. 8) (formed) on the posterior surface of the example refractive element, the example Kinoform lens structure having a (optical) power of −3.5 D. The example Kinoform lens structure has (e.g. consists of) 14 zones having a minimal zone radius of e.g. 0.8 mm and a step height of 6 μm. The example element inducing an increase of the chromatic aberration and the refractive element consist of the same example material and form an integral unit, the example material having an Abbe number of 42 and a refractive index of 1.52. The example element inducing an increase of the chromatic aberration, wherein the example increase of the chromatic aberration is an increase by a factor of two (over the chromatic aberration caused by the refractive element and the eye). For example, a positive refractive base with a power of 23.5 D was combined with a negative Fresnel lens (−3.5 D), which effectively doubled the pseudophakic eye's chromatic aberration. The second diffractive order (m=2) is used, and the diffractive surface consists of 14 Fresnel zone plates with a minimum zone radius of 0.8 mm. For the selection of m=2, the step height was 6 μm which may ensure the maximum efficiency. If, however, a different (e.g. positive or negative) diffractive order is to be used, the step height may be adjusted accordingly. For instance, for the selection of m=1, the first Fresnel zone radius may be 0.57 mm, and the step height may be 0.003 or 0.0033 mm. The example embodiment depicts a full-diffractive design with grating disposed on the back surface. Other embodiments that have the grating limited to the central-lens area (e.g., 3 mm or 4 mm) may allow for improved mesopic and scotopic vision. The example embodiment may be constructed using a material having a refractive index of 1.52 and an Abbe number of 42. A central lens thickness was e.g. assumed to be 1 mm.

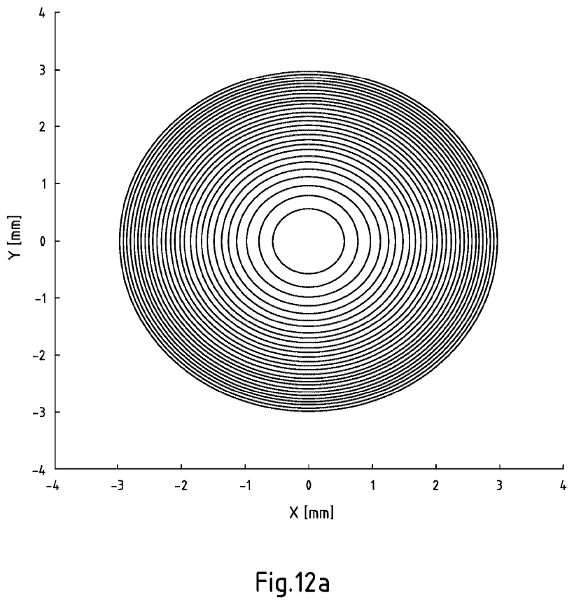

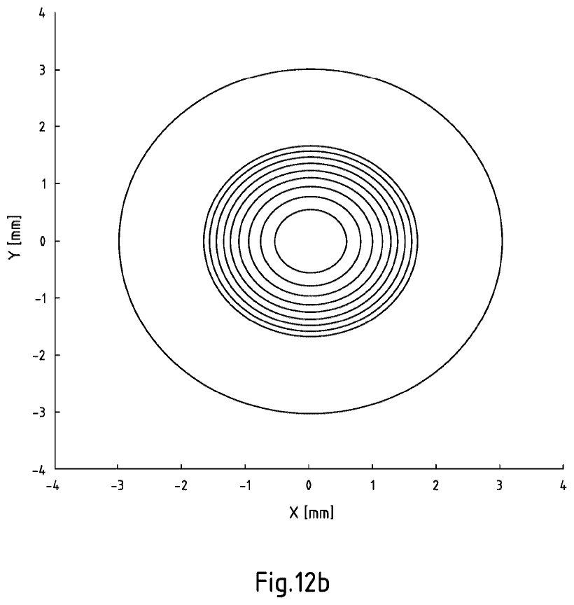

The diffractive-surface phase profile is presented in FIG. 8, showing a phase map (Modulo-2π) of a full diffractive surface. However, in other example embodiments the diffractive grating may e.g. be limited to the central-lens area (e.g., 3 mm or 4 mm, cf. e.g. FIG. 12b).

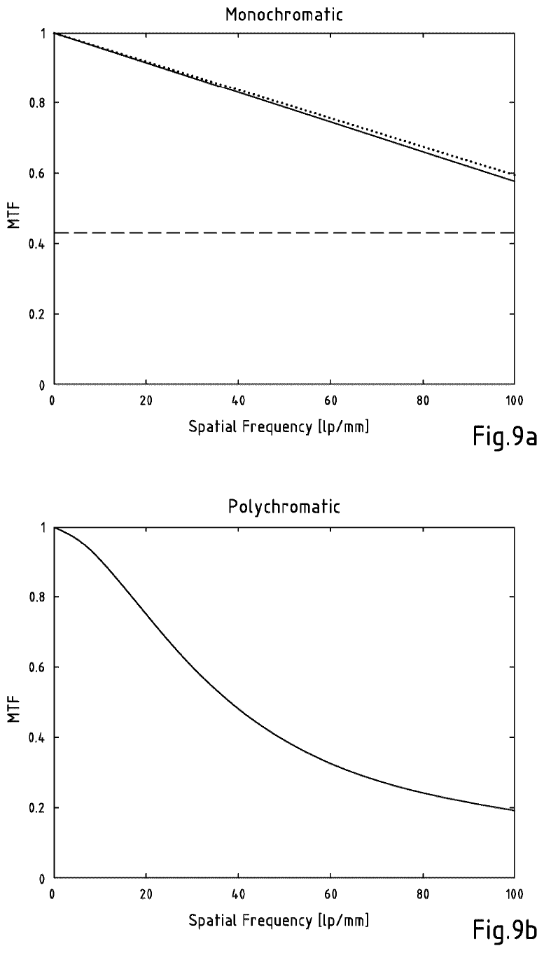

FIG. 9a and FIG. 9b show modulation transfer function (MTF) levels of one example embodiment with a 2-fold increase of longitudinal chromatic aberration in a model eye (the previously described example embodiment). Monochromatic (550 nm, FIG. 9a) and polychromatic (FIG. 9b) MTFs (solid lines) are shown up to 100 lp/mm. The dashed line (FIG. 9a) indicates the lowest (monochromatic) MTF at 100 lp/mm accepted for monofocal IOLs, the dotted line refers to a diffraction-limited modulation transfer (MT).

The simulated optical quality in the model eye meets the requirements of the manufacturing standards (ISO 11979-2) for monofocal lenses requiring a monochromatic (550 nm) modulation transfer function (MTF) at 100 lp/mm greater than or equal to 0.43. The described example embodiment exceeds this requirement with an MTF value of 0.58 and a nearly diffraction-limited performance (FIG. 9a). FIG. 9b shows a polychromatic MTF of the discussed embodiment.

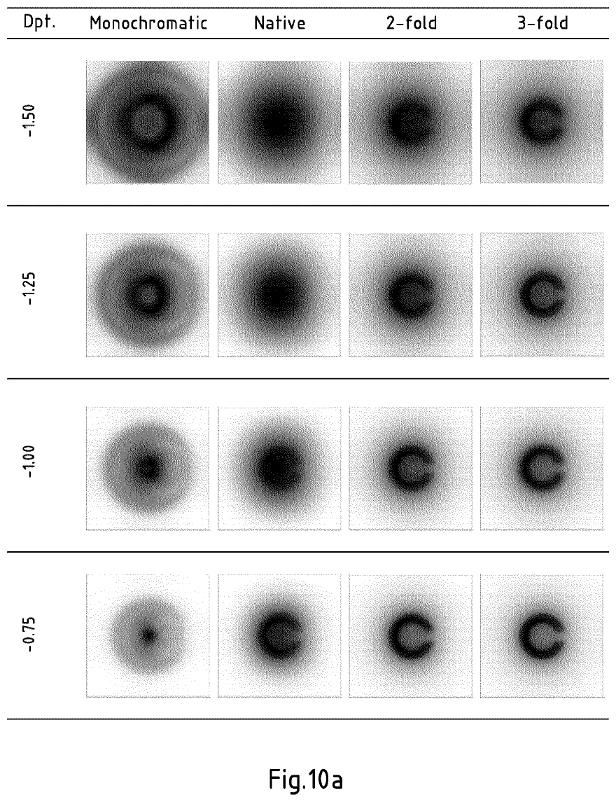

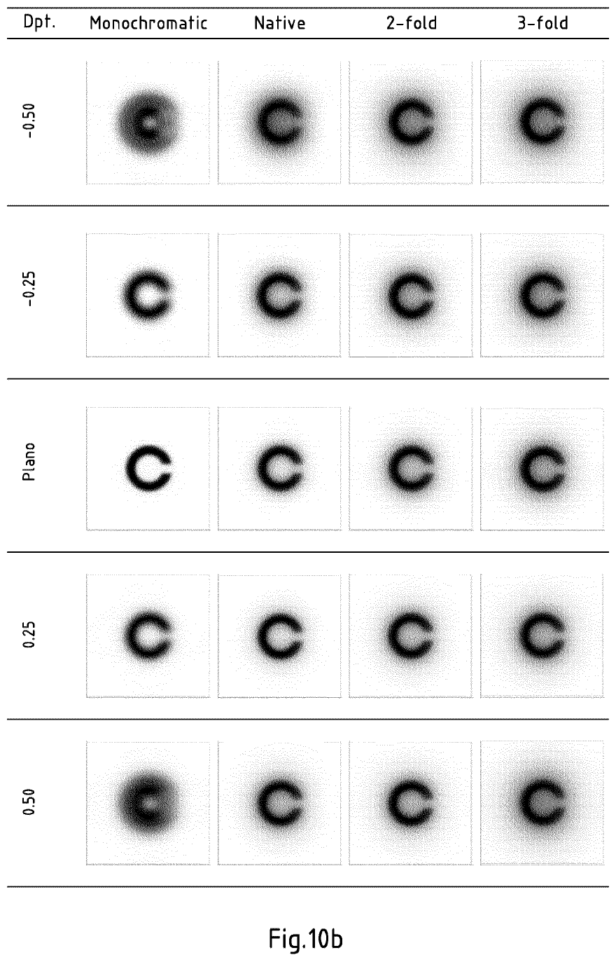

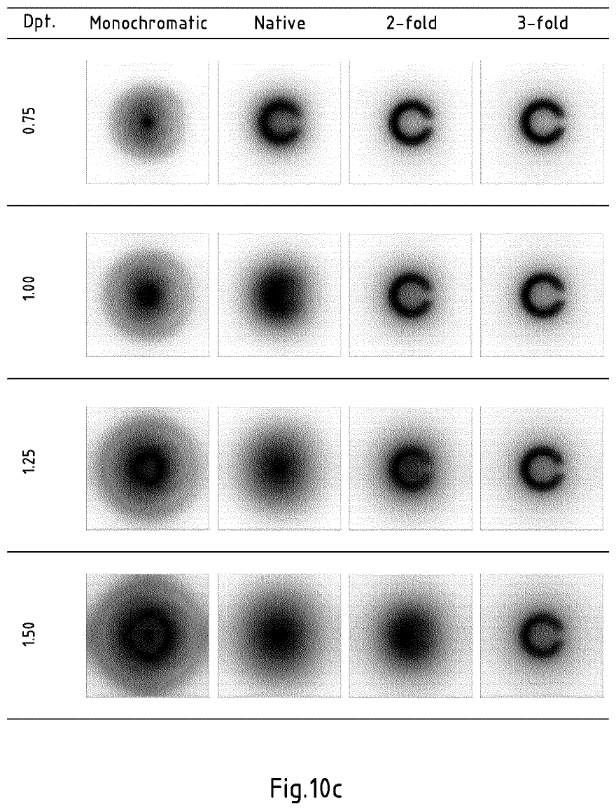

FIGS. 10a, 10b, and 10c show retinal-image simulations obtained in an ISO 11979-2:2014 model eye with above described example embodiments (with reference to FIG. 4a/b and FIG. 6a/b respectively), according to the first aspect featuring a 2-fold or 3-fold increase in LCA. Monochromatic and polychromatic (with spectral weighting) conditions were compared showing a substantial DoF increase due to chromatic aberration. Retinal-image simulations are presented in FIGS. 8a, 8b, and 8c with the corresponding defocus values. A 3-fold increase of LCA results in comparable image quality across a 2 D range (from −1 D to +1 D). An intentional myopic refractive target may expand a useful range of vision. Such an IOL also may offer a larger ‘landing zone’ which may allow to achieve better refractive outcomes after surgery.

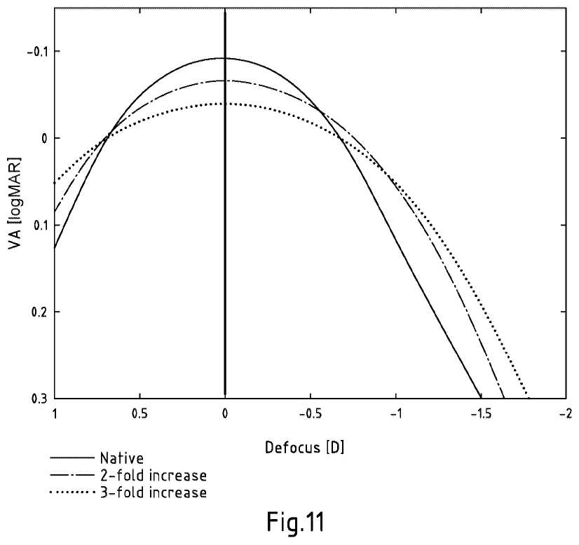

FIG. 11 shows simulated logMAR visual acuity of three conditions at the defocus range from +1 D to −2 D. The vertical dashed line indicates the position of the best far focus.

In the following, a quantification of the depth-of-focus extension is described:

The natural, 2- and 3-fold LCA conditions may be compared according to their image quality metrics at a 3-mm pupil. To this end, the area under the MTF (MTFa) may be obtained based on the following formula:

$$MTFa = {\sum }_{f = 1}^{f = 50 / d} MTF ( fd )$$

where d determines the sampling of the spatial frequency (f). The MTFa may be derived for each defocus position from +1D to −2D and converted to clinical visual acuity according to this model:

$$V A = a \cdot {MTFa}^{b} + c$$

The coefficients used in the calculations may be acquired from ANSI Z80.35-2018 (a=0.085, b=−1.0, and c=−0.21). In FIG. 11 the three conditions in terms of simulated visual acuity are compared. At the best far focus (0 D), the 2-fold LCA increase causes a mere change of visual acuity by 0.02 logMAR and 0.05 logMAR for the 3-fold increase. Still, the predicted visual acuity level is better than the normal population's average (i.e., 0.00 logMAR or 20/20 Snellen). The native-LCA model demonstrated a more substantial deterioration of the optical quality under defocus than the IOLs with increased LCA. For example, at 1.50D (67 cm visual distance), doubling the eye's LCA improved visual acuity by 0.07 logMAR, but for the triple amount, it was 0.11 logMAR.

FIG. 12a and FIG. 12b illustrate further example embodiments of an implantable lens comprising a refractive element causing chromatic aberration and an element inducing an increase of the chromatic aberration, wherein the element inducing the increase of the chromatic aberration comprises a diffractive structure, in particular the diffractive structure is a Fresnel structure, in particular a Kinoform lens structure, wherein the diffractive structure comprises a diffractive profile and wherein in particular the diffractive profile extends from a first Fresnel zone of the diffractive structure to an edge of the refractive element. The structures shown in FIGS. 12a, 12b may e.g. be used in conjunction with the example embodiment described with respect to FIGS. 8, 9a, and 9b.

In one example option illustrated in FIG. 12a, the design of the implantable lens is e.g. fully diffractive, where the diffractive structure (Fresnel rings) extend to the edge of the lens. In another example option illustrated in FIG. 12b, the design of the implantable lens is e.g. partially diffractive, where the diffractive structure (Fresnel rings) extend to a diameter of 3 mm, 4, or 4.5 mm, which may improve scotopic/mesopic vision and also may facilitate manufacturability. In FIGS. 12a and 12b the Kinoform lens structure is a negative Kinoform lens structure having a diffractive profile extending from the centre either to the edge of the lens (FIG. 12a) or to a certain distance from the centre (FIG. 12b). The Kinoform lens structure in FIGS. 12a and 12b being a (continuous) “sawtooth” surface profile having a local minimum in the centre (at radius 0 mm, i.e. X=Y=0 mm). The “sawtooth” maxima (depicted as concentric solid lines), where the profile reaches the step height, decrease with distance to the centre.

The expression “A and/or B” is considered to comprise any one of the following three scenarios: (i) A, (ii) B, (iii) A and B. Furthermore, the article “a” is not to be understood as “one”, i.e. use of the expression “an element” does not preclude that also further elements are present. The term “comprising” is to be understood in an open sense, i.e. in a way that an object that “comprises an element A” may also comprise further elements in addition to element A. Further, the term “comprising” may be limited to “consisting of”, i.e. consisting of only the specified elements. The expression “A and/or B” may also be understood to mean “at least one of A or B” or “at least one of the following: A or B”.

It will be understood that all presented embodiments are only examples, and that any feature presented for a particular example embodiment may be used with any aspect on its own or in combination with any feature presented for the same or another particular example embodiment and/or in combination with any other feature not mentioned. In particular, the example embodiments presented in this specification shall also be understood to be disclosed in all possible combinations with each other, as far as it is technically reasonable and the example embodiments are not alternatives with respect to each other. It will further be understood that any feature presented for an example embodiment in a particular category (method/apparatus/computer program/system) may also be used in a corresponding manner in an example embodiment of any other category. It should also be understood that presence of a feature in the presented example embodiments shall not necessarily mean that this feature forms an essential feature and cannot be omitted or substituted.

The statement of a feature comprises at least one of the subsequently enumerated features is not mandatory in the way that the feature comprises all subsequently enumerated features, or at least one feature of the plurality of the subsequently enumerated features. Also, a selection of the enumerated features in any combination or a selection of only one of the enumerated features is possible. The specific combination of all subsequently enumerated features may as well be considered. Also, a plurality of only one of the enumerated features may be possible.

The sequence of all method steps presented above is not mandatory, also alternative sequences may be possible. Nevertheless, the specific sequence of method steps as arranged in the wording of the claims or in the description above shall be considered as one possible sequence of method.

The subject-matter has been described above by means of example embodiments. It should be noted that there are alternative ways and variations which are obvious to a skilled person in the art and can be implemented without deviating from the scope of the appended claims.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An implantable lens comprising a refractive element causing chromatic aberration and an element inducing an increase of the chromatic aberration, wherein the element inducing the increase of the chromatic aberration comprises a diffractive structure, wherein the diffractive structure is a negative diffractive structure and wherein the diffractive structure is a Kinoform lens structure.

2. The implantable lens according to claim 1, wherein the increase of the chromatic aberration is an increase by a factor of at least two, preferably at least three, in particular a factor between three and seven.

3. The implantable lens according to claim 1, wherein the refractive element has a posterior and anterior surface, the posterior and/or the anterior surface having an aspheric or a spherical shape.

4. The implantable lens according to claim 1, wherein the element inducing the increase of the chromatic aberration increases the distance between a focus for red light having a wavelength of 650 nm and a focus for blue light having a wavelength of 450 nm, under standard room conditions, to 5 to 13 mm; and/or the element inducing the increase of the chromatic aberration increases the distance between a focus for red light of 656 nm and for blue light of 486 nm between 5 mm and 10 mm as measured in vitro or in immersion, wherein the immersion has a 1.333 refractive index; and/or the element inducing the increase of the chromatic aberration increases the distance between a focus for red light of 656 nm and for blue light of 486 nm between 0.8 mm and 1.3 mm as measured in vivo or in situ; and in particular the refractive element causes a chromatic aberration with a distance between the foci for red and blue light of 1 to 2 mm, as measured in vitro or in the immersion, and/or 0.4 to 0.6 mm, as measured in vivo or in situ.

5. The implantable lens according to claim 4, wherein the refractive element has a posterior and an anterior surface and the diffractive structure is on the posterior and/or the anterior surface.

6. The implantable lens according to claim 4, wherein the diffractive structure has a power of at most −1D, in particular the power is between −1D and −11D.

7. The implantable lens according to claim 4, wherein the diffractive structure comprises a diffractive profile and wherein in particular the diffractive profile extends from a first Fresnel zone of the diffractive structure to an edge of the refractive element or the diffractive profile extends from the centre of the refractive element to an edge of the refractive element.

8. The implantable lens according to claim 1, wherein the refractive element and the element inducing an increase of the chromatic aberration together comprise a diffractive-refractive zone centred on the centre of the refractive element, in particular the diffractive-refractive zone has a diameter from 3 mm to 4.5 mm.

9. The implantable lens according to claim 1, wherein the diffractive structure utilizes the first and/or second diffractive order.

10. The implantable lens according to claim 1, wherein the refractive element has a nominal power range of −5 D to 40 D.

11. The implantable lens according to claim 1, wherein the element inducing an increase of the chromatic aberration increases the chromatic aberration by at least 1D, preferably at least 2D, at a spectral range from 450 nm to 650 nm.

12. The implantable lens according to claim 1, wherein the element inducing an increase of the chromatic aberration and the refractive element consist of the same material, in particular the element inducing an increase of the chromatic aberration and the refractive element form an integral unit.

13. The implantable lens according to claim 1, wherein the Kinoform lens structure comprises a sawtooth surface profile, in particular superimposed on an anterior and/or posterior surface of the refractive element, wherein optionally the Kinoform lens structure may have a central zone lower with respect to a first diffractive step.

14. The implantable lens according to claim 13, wherein the sawtooth surface profile having a local minimum in the centre and in particular the distance between the maxima, where the sawtooth surface profile reached the step height, decreases with distance to the centre of the of the refractive element.

15. A method for producing an implantable lens according to claim 1, the method comprising etching and/or machining an element inducing an increase of the chromatic aberration on an anterior and/or a posterior surface of a refractive element.
